(19) United States
(12) Patent Application Publication
(10) Pub. No.: US 2026/0137282 A1
(43) Pub. Date: May 21, 2026

(54) CHRONIC OBSTRUCTIVE PULMONARY DISEASE AND/OR HEART FAILURE MONITORING BASED ON RESPIRATORY INFORMATION

(71) Applicant: BIOTRONIK SE & Co. KG, Berlin (DE)

(72) Inventors: Olaf SKERL, Bad Doberan (DE); Franziska WEGERICH, Berlin (DE); Andreas ARNDT, Berlin (DE)

(73) Assignee: BIOTRONIK SE & Co. KG, Berlin (DE)

(21) Appl. No.: 19/122,519

(22) PCT Filed: Oct. 13, 2023
(86) PCT No.: PCT/EP2023/078458

§ 371 Date: Apr. 18, 2025

(30) Foreign Application Priority Data
Oct. 24, 2022 (EP) 22203289.8

(51) Int. Cl. A61B 5/0205; A61B 5/0215; A61B 5/08

(52) U.S. Cl. CPC A61B 5/0205; A61B 5/0215; A61B 5/0816; A61B 2562/0247

(57) ABSTRACT

A method and apparatus for deriving respiratory information of a patient. The method includes the steps of acquiring a blood pressure signal and deriving respiratory information from the acquired blood pressure signal.

### CROSS-REFERENCE TO RELATED APPLICATIONS

This application is the United States National Phase under 35 U.S.C. § 371 of PCT International Patent Application No. PCT/EP2023/078458, filed on Oct. 13, 2023, which claims the benefit of European Patent Application No. 22203289.8, filed on Oct. 24, 2022, the disclosures of which are hereby incorporated by reference herein in their entireties.

### TECHNICAL FIELD

The present invention relates to methods, apparatuses and systems for deriving respiratory information from a blood pressure signal, particularly for monitoring patients suffering from heart and/or respiratory diseases.

### BACKGROUND

Patients suffering from heart diseases or with a known risk for heart diseases require regular monitoring of the current state of the heart disease. Therefore, in some cases such patients carry implantable sensors being capable of measuring the blood pressure. Heart diseases are commonly accompanied by comorbidities which additionally deteriorate the health state of the patient. However, such comorbidities cannot be monitored by the implantable blood pressure sensors and require additional diagnostic tools only available in a hospital and/or a medical practice. Beside the fact that additional hardware is required and that it creates an additional burden for heart failure patients to regularly visit a hospital and/or a medical practice, the exclusive monitoring of patients in a hospital and/or a medical practice only provides a sequential monitoring of the health state of the patient (and accompanied comorbidities). This commonly leads to a delayed determination of a deterioration of an existing comorbidity and/or the discovery of a comorbidity with the effect of unnecessary additional health constraints for the patient and/or even an acute exacerbation of the health state of the patient whereby a discovery of a comorbidity may occur rather by chance.

Therefore, there is a demand for improving the monitoring of patients with heart diseases.

This demand may at least in part be met by the aspects described herein.

The present disclosure is directed toward overcoming one or more of the above-mentioned problems, though not necessarily limited to embodiments that do.

### SUMMARY

According to a first aspect, a method for deriving respiratory information of a patient is provided. The method may comprise the steps of: acquiring a blood pressure signal and deriving respiratory information from the acquired blood pressure signal.

This provides the advantage that an (e.g. already implanted) blood pressure sensor may be used to derive information associated with the respiratory system of the patient. Known implantable pressure sensors typically measure the filling pressure of the heart, e.g., by means of sensors for measuring the pressure in the vena cava, the right ventricle, the pulmonary artery and/or the left atrium. These sensors are currently only being used for the determination of the cardiac filling pressures such that a potentially existing heart failure may be treated with drugs to affect the intravascular blood volume such that the patient does not experience any stasis symptoms and that the patient does not decompensate.

However, the inventors of the present invention have found that signals from said sensors can also be used to derive respiratory information, and thus respiratory information may be obtained without specific additional devices and/or on-site check-ups. Hence, known check-up methods (comprising non-portable devices/sensors) such as spirometry or spiroergometry, measuring the oxygen and/or carbon dioxide concentration of the blood or transcutaneous methods to control a potential oxygen therapy may become redundant. Instead, the respiratory system of the patient may be monitored without additional hardware.

The present invention expands the scope of the application of an (already) implanted blood pressure sensor of a patient (e.g. suffering from a heart disease) without requiring additional hardware which may otherwise e.g. only be available in a hospital and/or a medical practice. Additionally, the method may provide the advantage of a continuous monitoring of the health state of the patient without restricting a monitoring of the health state of the patient to (regular) check-ups only. This may allow an earlier detection/discovery of potential comorbidities (e.g. of the respiratory system) of a patient (associated with a heart disease) and/or an early detection if a deterioration of the health state of the patient occurs, e.g., by means of a deterioration of a comorbidity. Particularly, the information on the respiratory system of the patient may be helpful for diagnosing frequent comorbidities of patients with heart diseases, such as e.g. an increasing breathing resistance due to, e.g., a chronic obstructive pulmonary disease (COPD). It may, in particular, allow an early diagnosis, and correspondingly an early adoption of a potential therapy.

Moreover, the present invention further allows a simultaneous monitoring of more than one comorbidity which may increase the overall level of acceptance of an implant by a patient because a broader monitoring of a health state of the patient may be provided by means of a single implant. The monitoring of more than one comorbidity may, in particular, be motivated by the aspect that approx. 30 % of the patients suffering from a chronic heart failure (CHF) also suffer from COPD.

The blood pressure signal may relate to a (regular) sequence of systolic and diastolic extrema which may be associated with a pumping sequence of a heart.

The method may e.g. be performed by a processing system and/or a computer which receives the blood pressure signal from a respective sensor. Additionally, or alternatively, it may also be possible that the method is performed by an (implantable) sensor itself.

The blood pressure signal may, for example, comprise a signal measured by an implantable pressure sensor, preferably a pulmonary artery pressure (PAP) signal. The implantable pressure sensor may e.g. be implanted into the pulmonary artery such that the acquired blood pressure signal may comprise a PAP signal.

The pressure sensor may relate to an active sensor (e.g. comprising a dedicated (internal) power supply such as e.g. a battery) or a passive sensor (e.g. the sensor does not possess a dedicated power supply and/or the sensor may be supplied with power e.g. by means of an external excitation coil).

By acquiring a PAP signal, it may be possible to derive the intrathoracic pressure the PAP may be modulated with. By accessing the intrathoracic pressure, it may further be possible to derive a potential breathing resistance in the respiratory system (i.e. the airways such as e.g. the bronchia) of the patient (e.g. caused by an obstruction in the airways of the patient). Vice versa, any inhaling and/or exhaling of air may be mapped onto the intrathoracic pressure. The intrathoracic pressure may be mapped onto the measurable blood pressure, e.g. when measured in the pulmonary artery. Hence, it may be derived from the blood pressure signal.

The deriving of respiratory information may comprise deriving a respiration signal of at least one respiratory cycle of the patient. A respiration signal may e.g. relate to a blood pressure (p) vs. time (t) representation of the acquired blood pressure (preferably PAP) signal. It is understood that a respiration signal of a respiratory cycle comprises at least two data points (e.g. (t₁, p₁), (t₂, p₂) . . . ), preferably more than two data points.

A respiratory cycle may relate to the time interval during which an inhaling of air (i.e. filling the lung with fresh air; leading to an inspiratory minimum in the PAP) and an exhaling occurs (i.e. discharging the lungs; leading to an expiratory maximum in the PAP), until the next inhaling occurs. The number of inhaling sequences per time interval may also be referred to as the respiratory rate (also referred to as breathing rate hereinafter). The respiratory rate may be derivable from a duration of a respiratory cycle as its reciprocal value. It may be possible that a respiratory cycle comprises more than one heartbeat. In other words, a pulse rate may be higher than a breathing rate. It may be possible that more than one respiratory cycle is derived such that e.g. the PAP pressure vs. time may be derived over several seconds, several minutes, several hours, several days, etc.

Deriving a respiration signal may provide the advantage of identifying amplitude and/or duration of features mapped onto the blood pressure signal. The representation of the respiratory information in a respiration signal may further allow a display of the variation of a PAP pressure caused by respiration (e.g. to a member of a medical staff such as doctors, nurses, etc.) e.g. by means of a respiration vs. time diagram and/or may allow a simplified determination of certain events, e.g., mapped onto the pressure signal, which may occur in a periodic manner (e.g. a periodic exceedance of at least one parameter as it will further be described below).

The deriving respiratory information may include determining at least one parameter, e.g. associated with a COPD. A COPD issue may relate to a presence of a COPD and/or to a deterioration of a present COPD and/or simply a COPD event, for example.

The at least one parameter may preferably be determined from a time response (covering at least one respiratory cycle) of the respiratory information. The at least one parameter may e.g. relate to a duration of a respiratory cycle (and/or a breathing rate wherein the breathing rate may be the reciprocal of the duration of a respiratory cycle), the breathing depth (also referred to as breathing amplitude hereinafter; e.g. a (maximum) difference between inspiratory minima and the expiratory maxima for each respiratory cycle). The individual differences between the inspiratory minima and expiratory maxima may further be used to calculate average difference values between the inspiratory minima and the expiratory maxima which may be derived in a time interval of interest (which may exceed a duration of a respiratory cycle). Additionally or alternatively, it may be possible to derive said differences on any other time scale besides the above mentioned one.

Determining at least one parameter may allow a reliable determination of the presence and/or deterioration of, e.g., a COPD issue based on a quantitative variable (i.e. the at least one parameter).

The analysis may further comprise comparing the at least one determined parameter with a predetermined threshold. Dependent on the crossing of the said threshold a certain decision/conclusion may be based. The predetermined threshold may be associated with a breathing amplitude and/or a duration of a respiratory cycle (a breathing rate, respectively) and/or any other suitable parameter.

The usage of a predetermined threshold may e.g. provide the advantage that the definition of the threshold may tailored to the individual patient. There may be patients with naturally elevated breathing amplitude (i.e. patients regarded as healthy). Therefore, the definition of a respective predetermined threshold may be adapted to the individual health state of the patient, e.g., by choosing a rather high predetermined threshold as compared to patients which naturally show a rather low breathing amplitude. Said difference may e.g. be based at least in part on regular sports activities of the patient, pre-existing comorbidities, etc.

The method may further comprise determining whether the respiratory information indicates a chronic obstructive pulmonary disease (COPD). The decision whether the respiratory information indicates a COPD may at least in part be based on the time response of the PAP signal.

The indication may be based on a single surpassing of a predetermined threshold by at least one determined parameter. If a crossing of the threshold is determined, it may be concluded that a patient suffers from COPD and/or any other respiratory comorbidity. Alternatively and/or additionally, it may also be possible that the indication is based on a specified number of crossings (e.g. twice, three times, five times, ten times, etc.) of the threshold by the at least one determined parameter (e.g. the breathing amplitude and/or the breathing rate, etc.) within a certain time interval (e.g. within a single respiratory cycle, 1 s, 2 s, 5 s, 1 min, 1 hour, etc.). If the specified number of crossings is reached or exceeded, it may be concluded that a patient suffers from COPD and/or any other comorbidity. If the number of crossings is not reached, a patient may be regarded as not suffering from COPD and/or any other comorbidity.

It may be possible that a breathing amplitude is derived for each respiratory cycle and that a trend (e.g. a time response) of the breathing amplitude is derived based thereon. Hence, it may be possible to determine whether the breathing amplitude decreases, increases or remains at a constant level over time (e.g. over a week or month and/or any other suitable time scale). It may be possible to determine a health issue if a single crossing of a predetermined threshold occurs during the trend of the at least one derived parameter (e.g. the breathing amplitude) over time and/or that a number of crossings of the trend of the at least one determined parameter in a certain time interval (as described above) is needed, which may e.g. be applicable if the trend shows an oscillatory behavior over time. Similarly, as outlined above, the health issue may for example be COPD.

It may be possible that the breathing amplitude and/or the trend of the breathing amplitude and/or any other suitable parameter associated with the respiratory system of the patient may be evaluated such that a weighting factor may be assigned to the respective parameter. The weighting factor may be understood as a parameter which may indicate how relevant a derived respiratory information may be seen for the health status of the patient.

The derivation of a trend may also be possible for the duration of a respiratory cycle (as described for the breathing amplitude above). Predetermined thresholds may equally be applicable to a trend of the duration of a respiratory cycle as described above.

It is further noted that any crossing of a certain value includes the exceedance and the undershooting of the certain value.

The method may further include acquiring activity and/or posture data. Activity data may relate to data measured by e.g. an accelerometer (or any other suitable device) which may indicate whether a patient is currently at rest, walking, jogging, running, etc. Posture data may relate to data indicating whether a patient is lying (e.g. the patient is in a horizontal position), sitting, standing (i.e. the patient is in an upright/vertical position), etc. The activity and/or posture data may e.g. be acquired from a suitable device contained in the pressure sensor and/or may be acquired by an additional implant (e.g. a loop recorder, a heart monitor and/or a therapy implant) and/or may be acquired by any other device e.g. carried by the patient (e.g. a patch, a smartphone, a smartwatch, a wearable accelerometer, etc.).

The activity data may be used to generate an activity index and/or profile characterizing the overall physiological activity of the patient.

By considering the current activity and/or posture data in addition to the respiratory information (e.g. correlating it) an additional parameter to characterize the health status of the patient can be provided. Thus, a more accurate prediction/indication whether a patient suffers from COPD may be achieved.

The method may further comprise correlating the respiratory information with corresponding activity and/or posture data. Both a breathing amplitude and a duration of a respiratory cycle may depend on the current activity and/or posture of the patient, for example. Notably, patients suffering from a heart disease may tend to exhibit shortness of breath when performing (exhausting) sports activities (such as e.g. running) or even simply when walking. This may be accompanied by a decrease in breathing volume which may be manifested in a decrease of the breathing amplitude and/or an increase in breathing rate. Additionally, or alternatively, shortness of breath may even occur in certain postures such as e.g. lying (sleeping). By correlating the respiratory information and the activity and/or posture data, it may be possible to determine whether a patient suffers from a heart disease, an existing heart disease/comorbidity is worsening and/or a new comorbidity is developing.

The activity and/or posture data and the correlated blood pressure signal may both be acquired during the same time interval. The acquisition of the blood pressure signal may be synchronized with the acquisition of the activity and/or posture data. Alternatively, it may be possible that the acquisition of the blood pressure signal is triggered by a detected activity and/or a posture change.

The method may further comprise comparing first respiratory information of a first time interval associated with first activity and/or a first posture, with second respiratory information of a second time interval associated with a second activity and/or second posture. The comparison of two time intervals may allow a derivation of the change of the respiratory information over time. It may be possible, that a patient does intensive exercise during the first time interval which is suddenly stopped as the second time interval begins. This may allow a conclusion on the regeneration/recovery time of the patient after said intensive activity (e.g. a sports activity such as e.g. running, cycling, etc.) and/or a posture change (e.g. standing up). After an intensive activity, a pulse rate may be increased and/or a shortage of breath may be more dominantly pronounced immediately after the activity but may decay during a certain time which may be understood as a recovery time of the patient (e.g. during a certain time at rest). This recovery time may be observable e.g. in a change in breathing amplitude and/or duration of a respiratory cycle after an activity period and/or posture change.

As an alternative, it may also be possible that a change of activity occurs during a transition from the first time interval to the second time interval. A change of activity may relate to a change from an intensive activity (e.g. fast running) to a less intensive activity (e.g. jogging) and vice versa. Similarly, as outlined above (for the sudden stop of an activity), it may again be possible to derive the respective recovery time of the patient.

It may be possible that the first time interval and the second time interval are adjacent time intervals (e.g. the beginning of the second time interval may be equal to the end of the first-time interval). However, it may also be possible that the second time interval is a certain period of time distant to the first time interval. In other words, the second time interval may e.g. begin 1 s, 10 s, 1 min, 1 hour, etc. after the end of the first time interval.

The comparison of two time intervals may allow a characterization of, e.g., the recovery time of a patient and may thus be used as an additional parameter on which a determination may be based whether a patient suffers from a comorbidity. The recovery time of the patient may e.g. be prolonged if a patient suffers from COPD and/or a heart disease. Moreover, the shortness of breath may be intensified (e.g. after an intensive activity) and the time it remains at an elevated level may be longer for patients with a comorbidity as compared to patients which are considered as healthy.

The method may further comprise determining whether respiratory information indicates a heart disease.

A heart disease may e.g. relate to a CHF issue. A CHF issue may be understood as the presence of CHF and/or a deterioration/decompensation of a present CHF. A heart disease may be understood as a behavior of the heart which deviates from the behavior of a healthy heart.

The indication may preferably be based on the time response of the PAP signal. The decision, whether a heart disease is indicated or an existing heart disease has been deteriorated may be based on the crossing of a predetermined threshold (wherein the predetermined threshold relates to a threshold duration of the respiratory cycle). In an example, a patient suffering from a CHF issue may tend to exhibit shortness of breath. Therefore, the average respiratory cycle of a patient with a CHF issue may tend to exhibit a shorter respiratory cycle compared to a patient which is considered as healthy or stable. Therefore, a predetermined threshold may be chosen such that any duration of a respiratory cycle shorter than the threshold duration may be interpreted as an indication for a heart disease. Additionally, if such shortness of breath occurs already at moderate to low activity (e.g. as determined by correlating the respiratory information with activity and/or posture data) or there is a long recovery time (e.g. as determined as outlined above), this may be used as an indication and/or a confirmation of a heart issue, such as CHF.

The method may further comprise calculating a score value, indicative of COPD and/or a heart issue (such as CHF), based at least in part on the respiratory information.

A score value may refer to a quantitative number which may be applied to determine whether a patient suffers from COPD and/or a heart issue. If the number crosses a predetermined threshold, it may be concluded that a patient suffers from COPD and/or a heart issue. The number may e.g. also be expressed in terms of a probability (between 0% and 100%) a patient may suffer from COPD and/or a heart issue.

The calculation of the score value may be based on one or more of a blood pressure, a PAP, a correlation thereof with activity and/or posture data, a change of the blood pressure and/or PAP after a change of activity and/or a change of a posture, a pulse rate (which may be derived from a PAP), a body temperature (e.g. measured by the (implanted) pressure sensor), the inclination of the body (preferably the torso) during sleeping (e.g. patients suffering from a heart disease may tend to sleep with an elevated torso which may be expressed in a different inclination of the torso as compared to healthy patients (when lying/sleeping)) and/or any other suitable parameter. The calculation of the score value may additionally comprise weighting factors.

The score value may be calculated as a single number only at one distinct time. Alternatively it may be possible that a score value is calculated on a periodic basis (e.g. for each respiratory cycle, once every second, once every minute, once every hour, once every day, etc.) and/or on an aperiodic basis (e.g. only if an external trigger input is received by the pressure sensor and/or by an external device suitable of performing the calculation of a score). If multiple score values are calculated (e.g. for different time intervals and/or respiratory cycles), it may be possible to derive a trend of the score value over time. It may be possible to use an increase of the score value over time to estimate a disease progression. It may further be seen advantageous to apply further statistical analysis to the one or more scores (such as e.g. the derivation of a standard deviation) to confirm a potential indication that a patient suffers from COPD and/or a heart issue.

The calculation of a score may provide the advantage of providing a quantitative measure of the current health state of the patient (e.g. if the patient suffers from a COPD and/or a heart issue such as CHF). Moreover, it may be possible to use the trend of the calculated score values to determine how the health status of the patient may have changed over time. This may e.g. be of relevance if an initially calculated single score value does not allow a reliable conclusion whether a patient suffers from a COPD and/or a heart issue which may require a more detailed monitoring of the health state of the patient over time. The change of the score value over time may also be used to rate therapy results or to control therapy options.

Another aspect of the present invention relates to an apparatus for deriving respiratory information of a patient. The apparatus may comprise means for acquiring a blood pressure signal and means for deriving respiratory information from the acquired blood pressure signal.

The means for acquiring a blood pressure signal may comprise means for receiving the blood pressure signal from an implantable pressure sensor, preferably a pulmonary artery pressure (PAP) sensor. The PAP sensor may not be part of the apparatus but may be in communication with the apparatus, e.g. by means of a wireless communication (such as e.g. Bluetooth, near field communication (NFC), Wi-Fi, etc.) and/or by means of a wired connection. It may be provided that the apparatus receives a continuous data stream (e.g. comprising realtime data associated with a measured blood pressure) and/or that the apparatus receives the data in blocks (e.g. comprising measurement intervals of 1 s, 10 s, 1 min, 1 hour, etc.).

The apparatus may be a device carried by the patient (e.g. a smartphone, a smartwatch, a smart patch etc.). Additionally, or alternatively, the apparatus may also relate to a hospital and/or point of care device. By means of the apparatus, which may be equipped with an App, it may be in particular possible to indicate to the patient and/or a member of a medical staff that a COPD and/or CHF issue has been detected. It may be possible that the app indicates a COPD and/or a CHF issue by means of a push notification such that a patient may be triggered to contact a member of a medical staff for further consultation. It may also be intended that the data is additionally forwarded by the apparatus to a remote system (e.g. a cloud-based server system, a hospital information system, etc.) to supply additional redundancy for the data storage and for further processing.

In some examples, also a system may be provided comprising the above apparatus and a separate implantable blood pressure sensor (such as a pulmonary artery pressure sensor).

However, it is also possible that the apparatus may be comprised by an implantable (blood) pressure sensor, preferably a pulmonary artery pressure (PAP) sensor. In such an embodiment, any of the above-mentioned means may be implemented in a single implantable pressure sensor. The implantable PAP sensor may be configured for implantation via a catheter.

Additionally, the apparatus may comprise means for performing any of the above-mentioned analysis steps.

It is noted that the analysis steps as described herein may include all aspects described herein, even if not explicitly described as analysis steps but rather with reference to an apparatus (or device). Moreover, the apparatuses as outlined herein may include means for implementing all aspects as outlined herein, even if these may rather be described in the context of analysis steps.

Another aspect of the present invention relates to a computer program comprising code which performs the method as described above when executed.

The functions/method steps described herein may generally be implemented in hardware, software executed by a processor, firmware, or any combination thereof. If implemented in software executed by a processor, the functions may be stored on or transmitted to a computer-readable medium as one or more instructions or code. Other examples and implementations are within the scope of the disclosure and appended claims. For example, algorithms described herein can be implemented using software executed by a processor, hardware, firmware, hardwiring, or combinations of any of these. Features implementing functions may also be physically located at various positions, including being distributed such that portions of functions are implemented at different physical locations. Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both nonvolatile computer storage media and communication media including any medium that facilitates transfer of a computer program from one place to another. A nonvolatile storage medium may be any available medium that can be accessed by a general purpose or special purpose computer. By way of example, and not limitation, nonvolatile computer-readable media may comprise buffered RAM, ROM, EEPROM, Flash Memory, compact disk (CD) ROM or other optical disk storage, magnetic disk storage or other magnetic storage devices, or any other nonvolatile medium that can be used to carry or store desired program code means in the form of instructions or data structures and that can be accessed by a general-purpose or special-purpose computer, or a general-purpose or special-purpose processor. Combinations of the above are also included within the scope of computer-readable media.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The following figures are provided to support the understanding of the present invention:

FIG. 1: Illustration of a typical PAP signal vs. time;

FIG. 2: Illustration of a typical PAP signal vs. time and a derivation of respiratory cycles vs. time;

FIG. 3: Illustration of a single respiratory cycle derived from a PAP signal.

### DETAILED DESCRIPTION

The following detailed description outlines possible exemplary embodiments of the invention.

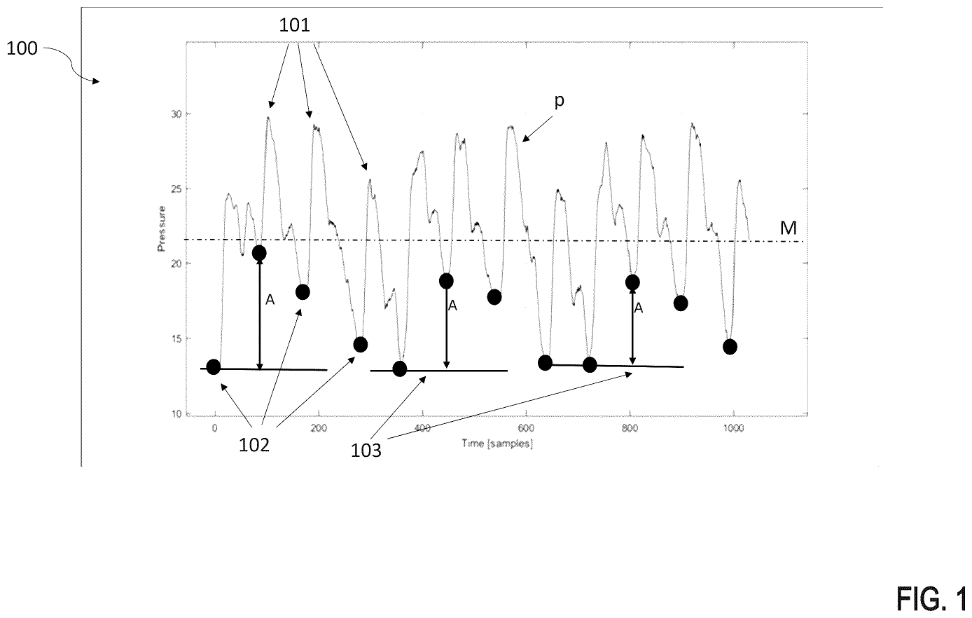

FIG. 1 shows an exemplary illustration 100 of a typical PAP (as a representative of a blood pressure) vs. time, as acquired by means of an (implanted) pressure sensor, preferably implanted in a pulmonary artery of a patient via a catheter. The output of the pressure sensor may be sampled with a respective data acquisition system. The typical PAP signal p may be a sequence including systolic maxima 101 and diastolic minima 102. A pair of a systolic maximum 101 and a diastolic minimum 102 may be understood as a single heartbeat. The sequence of systolic maxima 101 and diastolic minima 102 may be arranged around a certain mean value M (e.g. obtained from an averaging of the illustrated PAP signal p over the displayed time interval (with respect to the abscissa of the diagram)).

The PAP signal p may be acquired by sampling the pressure at a sampling frequency which exceeds the pulse rate of the patient such that additional features which may be mapped onto a classic pulse signal (i.e. which occur on shorter time scales such as e.g. the transition from a diastolic to a systolic pressure sequence and vice versa) may be captured. It may be possible that said additional features occur on shorter time scales as compared to the time scale of a heartbeat, such as e.g. the build-up of a systolic maximum 101. Assuming a pulse rate of 60 bpm at rest, the sampling frequency may e.g. exceed 2 Hz, 4 Hz, 6 Hz, etc. However, it may also be possible to choose a sampling frequency which is significantly higher (e.g. 100 Hz, 1 kHz, etc.) to obtain a more detailed sampling (e.g. with respect to any kind of features occurring therein) of a heart beat and/or a respiratory cycle. The PAP signal p may additionally be modulated by features associated with the respiratory system of the patient such as inhaling and exhaling of air.

A respiration cycle consists of one inhaling phase succeeded by one exhaling phase and may comprise one or more heart beats. The respiration cycle may be mapped on the acquired pressure signal p as described above and produces periodic variations of diastolic minima 102, respectively. Inhaling phase is indicated by a local minimal value 103 of diastolic pressure minima whereas exhaling phase is indicated by a local maximum of diastolic pressure minima. Based thereon, a breathing amplitude A may be defined which may indicate the maximum difference between diastolic pressure minima at exhaling and diastolic pressure minima at inhaling. Breathing amplitude may also be evaluated based on systolic pressure maxima in an analogous manner.

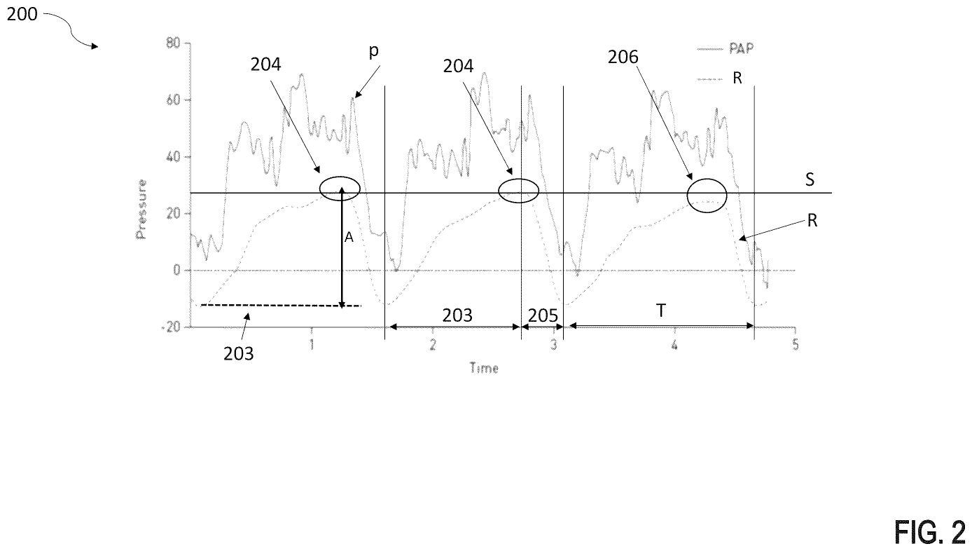

FIG. 2 shows an exemplary illustration 200 of a PAP signal p vs. time and a derivation of a respiration signal R. PAP signal p may be identical to the PAP signal p as described above with respect to FIG. 1.

It may be possible to derive a signal R from said PAP signal p by removing the heartbeat variation with the help of sufficient filter methods known in the art. The remaining respiration signal may allow a determination of a breathing amplitude A with less disturbances. It may further be noted that the PAP mean value has been subtracted from the respiration signal R for increased clarity when simultaneously displaying PAP signal p and respiration signal R in a single diagram.

Additionally or alternatively, it may be possible to derive a respiration signal R by determining all diastolic minima or systolic maxima in the original PAP signal p, as illustrated in FIG. 1, and interpolating the respective values. It may be possible to define a threshold S. Threshold S may be understood as a threshold which may be used to determine whether a patient suffers from a COPD issue. If a patient suffers from a COPD issue, an obstruction in the airways may lead to an increased expiratory pressure when exhaling and a decreased inspiratory pressure when inhaling such that the breathing amplitude A may be increased for patients suffering from COPD. If a patient has already been diagnosed with COPD, a deterioration of (existing) COPD may be indicated as an increase of breathing amplitude A. The breathing amplitude A may reach its maximum directly after starting to exhale when the lungs are still (almost) fully filled.

The increase of the breathing amplitude may at least in part be based on an obstruction in the airways of the patient. Said obstruction may lead to an increased pressure difference between the trachea and alveoli as the airways may be understood as e.g. partially blocked due to the obstruction. This may lead to a decreased intrathoracic inspiratory pressure and an increased intrathoracic expiratory pressure. These effects on the intrathoracic pressure may further lead to an increased intrathoracic pressure amplitude (i.e. the difference between the PAP during a full inhalation of air and a full exhalation of air) which may then be mapped onto the PAP signal p.

For better understanding, time intervals 203 denote the time intervals during which an inhaling occurs whereas time interval 205 exemplarily denotes a time interval during which an exhaling occurs. A combination of time intervals 203 and time intervals 205 denotes the duration T of a respiratory cycle.

An exceeding of the threshold S may indicate a COPD issue. It may be possible that an exceedance 204 of threshold S occurs (reproducibly) several consecutive times (i.e. in several subsequent respiratory cycles) and may even occur during each respiratory cycle. However, it may also be possible that there are some respiratory cycles during which the threshold S is not exceeded 206. The occurrence of at least one exceedance or at least a minimum number of exceedances in a predetermined time interval may trigger an indication of COPD.

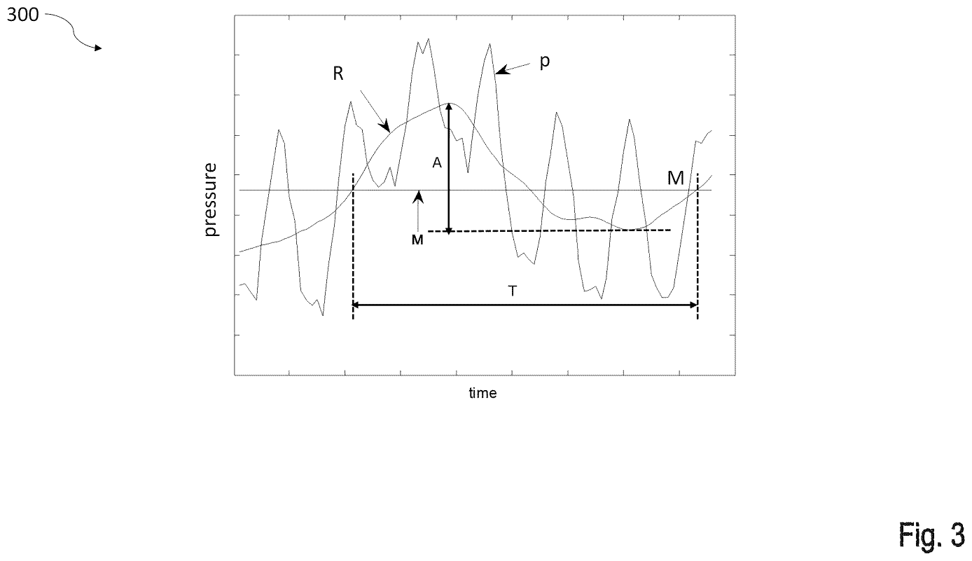

FIG. 3 shows an exemplary illustration 300 of a single respiratory cycle R with period T. A respiration signal R may be derived from the PAP signal p, wherein the signal R may be identical to the respiration signal R as described above with reference to FIG. 2. In particular, respiration signal R may be identically derived from the PAP signal p as described above with reference to FIG. 2.

In FIG. 3, PAP signal p and the derived respiration signal R (no offset has been subtracted in this case) may be arranged about a mean value M. Mean value M may be identical to mean value M as described above with reference to FIG. 1.

It may be possible to define a breathing amplitude A for each respiratory cycle, wherein the breathing amplitude A may be identical to breathing amplitude A as described above with respect to FIG. 2. It may be the difference between a maximum value and a minimum value of the respiration signal R derived from the pressure signal p within the respiratory cycle.

A respiratory cycle may be defined by its duration T. It may be defined as the time between a first crossing of R of the value M with a positive slope and the next such crossing.

An aspect of the present invention addresses the determination whether a patient suffers from a CHF issue which may be indicated by a shortage of breath (in particular if a patient performs intensive activities or performs a posture change) which may be observable as a decrease of the duration T of a respiratory cycle and/or the amplitude A.

As outlined above, it may be preferably to correlate the duration T of a respiratory cycle with activity data and/or posture data to obtain means for an interpretation of the duration T of a respiratory cycle. It may thus be possible to e.g. define a predetermined threshold (e.g. a certain predetermined threshold duration T of a respiratory cycle), that may e.g. depend on the activity level and/or posture, which when exceeded, may be used as an indication to determine that a patient suffers from a COPD issue. The determination whether a patient suffers from a COPD issue may generally be based on a correlation of one or more parameters associated with PAP signal p and activity and/or posture data.

In the past, for monitoring patients with a known COPD it was commonly recommended to regularly perform spirometry examinations and/or pulse oximetry measurements to determine respiration parameters. These known examination methods typically do not include the current activity and/or posture and/or posture changes of the patient and their potential effects on the respiratory system of the patient. It is only known that various physiological parameters may be linked on a statistical basis. However, the immediate reactions of the physiological parameters to a change of activity and/or posture are neglected.

However, according to the present invention, it may be possible to derive the duration T of a respiratory cycle as a mean duration T of many acquired respiratory cycles (e.g. over the course of 24 h and/or over a night and/or any other suitable duration). It may be possible to correlate said mean duration T with a mean activity, wherein the mean activity may be derived from acquired activity data (e.g. over the course of 24 h and/or over a night and/or any other suitable duration). Additionally, it may also be possible to incorporate a mean posture of the torso of the patient, a mean pulse rate (e.g. obtained from PAP signal p), a difference between the duration T of a respiratory cycle in a lying posture of the patient compared to a duration T of a respiratory cycle in an upright (standing) posture of the patient, a difference between the duration T of respiratory cycles when acquired for different pre-defined levels of activity (e.g. different running/jogging speeds, etc.) and/or any conjunction/correlation between changes in the PAP signal p and the duration T of a respiratory cycle for any kind of activity and/or posture changes. Additionally or alternatively, it may also be possible to derive a ratio of the duration T of a respiratory cycle with a pulse rate. It is noted that these parameters are only mentioned exemplarily to support the understanding of the invention. However, any other parameter, not mentioned above, may also be incorporated in the determination whether a patient suffers from a CHF or a COPD issue.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A method for deriving respiratory information of a patient, comprising:
acquiring a blood pressure signal; and
deriving respiratory information from the acquired blood pressure signal.

2. The method of claim 1, wherein the blood pressure signal comprises a signal measured by an implantable pressure sensor, preferably a pulmonary artery pressure, PAP, signal.

3. The method of claim 1, wherein the deriving of respiratory information comprises deriving a respiration signal of at least one respiratory cycle of the patient.

4. The method of claim 1, further comprising determining at least one parameter from the respiratory information.

5. The method of claim 4, wherein the method further comprises comparing the at least one parameter with a predetermined threshold.

6. The method of claim 1, further comprising:
determining whether the respiratory information indicates a chronic obstructive pulmonary disease, COPD.

7. The method of claim 1, further including acquiring activity and/or posture data.

8. The method of claim 7, further comprising correlating the respiratory information with corresponding activity and/or posture data.

9. The method of claim 7, further comprising comparing first respiratory information of a first time interval associated with a first activity and/or a first posture with second respiratory information of a second time interval associated with a second activity and/or a second posture.

10. The method of claim 1, further comprising:
determining whether the respiratory information indicates a heart disease.

11. The method of claim 1, further comprising:
calculating a score, indicative of a chronic obstructive pulmonary disease, COPD, and/or a heart issue, based at least in part on the respiratory information.

12. An apparatus for deriving respiratory information of a patient, comprising:
means for acquiring a blood pressure signal; and
means for deriving respiratory information from the acquired blood pressure signal.

13. The apparatus of claim 12, wherein the means for acquiring the blood pressure signal comprises means for receiving the blood pressure signal from an implantable pressure sensor, preferably a pulmonary artery pressure, PAP, sensor.

14. The apparatus of claim 12, wherein the apparatus is comprised by an implantable pressure sensor, preferably a pulmonary artery pressure, PAP, sensor.

15. A computer program comprising code which performs the method of claim 1 when executed.
